(19) United States
(12) Patent Application Publication
(10) Pub. No.: US 2022/0355076 A1
(43) Pub. Date: Nov. 10, 2022

(54) FIXATION MEANS FOR MEDICAL PERFORATION DEVICES

(71) Applicant: Bedal NV, Diepenbeek (BE)

(72) Inventors: Alexander Van Damme, Zoersel (BE); Simon Callaerts, Diepenbeek (BE)

(21) Appl. No.: 17/621,394

(22) PCT Filed: Jun. 29, 2020
(86) PCT No.: PCT/EP2020/068201

§ 371 Date: Dec. 21, 2021

(30) Foreign Application Priority Data
Jun. 27, 2019 (EP) 19182944.9
Feb. 13, 2020 (EP) 20157256.7

(51) Int. Cl. A61M 25/02; A61M 39/10

(52) U.S. Cl. CPC A61M 25/02; A61M 39/1011; A61M 2025/0266; A61M 2025/024; A61M 2025/028

(57) ABSTRACT

The present invention relates to means for fixating catheters (24), which are used in healthcare and in particular to means for fixating such catheters to a support (1).

### FIELD OF THE INVENTION

The present invention relates to means for fixating catheters, which are used in healthcare and in particular to means for fixating such catheters to a support.

### BACKGROUND TO THE INVENTION

In healthcare, a catheter is used to provide an access to the human body for drainage of a bodily fluid or for delivery of medicinal drugs, parenteral nutrition, blood or blood components or other liquids. A catheter is typically used for patients that are ill or during the performance of surgical procedures. Depending on the type of liquid that needs to be delivered or evacuated and sometimes also depending on the patient or his/her medical condition, different catheters and different application methods can be selected.

A first type of catheter is the peripheral venous catheter. This catheter is inserted in a peripheral vein. A second type of catheter is a midline catheter. Such a catheter typically is between 8 and 25 cm long and is often placed in an upper arm vein. A third type of catheter is a central venous catheter which is placed into a large vein.

Other types of catheters include continuous peripheral nerve block catheters and epidural catheters. These catheters can be applied at several places on the human body, such as for example in the breast area of a patient.

A large number of patients needs to make use of catheters, such as for example central catheters, for a long period, e.g. weeks or months. It often is preferred to make use of a same catheter for a longer time period, as correctly positioning a catheter is time consuming and as replacing or re-introducing a catheter into the human body typically results in additional risks for infections and additional pain for the patient during installing. Guidelines on catheter management also indicate that the number of manipulations of the catheter should be minimized.

A disadvantage of the use of a catheter is that it often limits the patient in actions they can take. For example, excessive movement whilst a catheter or part thereof is applied to the human body may be difficult or impossible as the position of the catheter within the body should under all circumstances remain fixed. Accidental catheter removal, upon the exertion of a pulling force should be avoided. A change in position of the catheter (e.g. catheter dislocation or migration) may prevent proper operation and, in some circumstances, could even lead to dangerous or life-threatening situations. Adequate fixation could prevent critical situations and could allow for these movements to be performed in safety. The positive effects of using a catheter stabilization device on the lifetime of the catheter, the decrease in the amount of unscheduled restarts and the decrease in the number of complications (e.g. phlebitis, extravasion or infections) have been demonstrated in the scientific literature.

Known medical devices for securing catheters are commercially available. However, these medical devices are often suited only for one catheter diameter or type, or for a limited range thereof, such that several different medical devices need to be purchased and used for different catheters.

Besides that, the fixation means are often unwieldy and bulky. A number of known medical devices for securing catheters do not deliver a significant increase in the fixation force that is required to dislocate the catheter. A number of known medical devices do not prevent small movements of the catheter in longitudinal direction, also known as micropistoning, leading to increased risk of infections. A number of known medical devices do not provide a placement of the catheter under an angle between 5° and 10° that is beneficial to the functioning of the catheter.

Also, it is of great importance to tightly secure the catheters against the patient's skin thereby inhibiting substantially all movement of the catheter in order to reduce the risk of for example bloodstream infections. A number of known medical devices fail to do so.

Some examples such as the granted patents U.S. Pat. Nos. 9,463,303 and 8,052,652, require fixation straps or filaments for fixating the catheters. The present invention disregards the use of such fixation straps or filaments and therefore tackles the unwieldy aspect of these known medical devices.

Other examples include for example the medical device as disclosed in U.S. Pat. No. 8,241,253, which describe attachment surfaces for attaching the medical article and permitting the catheter to be reoriented in multiple positions by rotation of the catheter relative to the support structure. The present invention makes it impossible for the catheter to be rotated or otherwise moved relative to the medical device, thereby reducing the risk of for example bloodstream infections once the catheter is introduced into the human body.

Furthermore, known medical devices such as WO2019063847, US2010324491, US2014142538, US2012041377 and US2014188078 are able to fixate different types of medical articles. However, none of these devices are able to tightly secure different types and sizes of luer-locks of medical articles with a single device.

There is thus still a need in the art for a means for tightly securing the luer-lock portion of medical articles which addresses some or all of the issues outlined above.

### SUMMARY OF THE INVENTION

It is an object of the present invention to provide good means for securing a catheter to a support (e.g. a human body). This objective is accomplished by a device according to the present invention.

It is an advantage of particular embodiments of the present invention that a catheter can be well secured with little or no movement within the medical device.

It is an advantage of particular embodiments of the present invention that the medical device can receive catheters of varying sizes (e.g. diameter).

It is an advantage of particular embodiments of the present invention that the medical device can receive different types and sizes of catheters and/or luer-locks.

Unless provided otherwise, the term “luer-lock” should be understood as standardized fittings that come in different shapes and sizes and are designed to create a leak-free connection between a male end (thread syringe end portion) and a female end (tabbed hub of a needle) of a medical article. When securely joined, a leak-free connection between the syringe and the needle is realized. On the outside, luer-locks may have a generally flat surface or a generally ribbed surface. In most cases, the luer-lock portion of the medical article forms an abutment, contributing to the irregular shape of the medical article. In the present invention, the medical device Is adapted to fixate the luer-lock portion of the medical article.

Therefore, it is an advantage of particular embodiments of the present invention that the medical device can fixate at least a portion of different types and sizes of luer-locks.

It is an advantage of particular embodiments of the present invention that the medical device can stabilize the catheter without applying an adhesive on the catheter

It is an advantage of particular embodiments of the present invention that a catheter can be secured to a living creature or to an inanimate object.

It is an advantage of particular embodiments of the present invention that the medical device may conform to the support. It is a further advantage of embodiments of the present invention that a considerable level of user comfort can be achieved.

It is an advantage of particular embodiments of the present invention that the catheter can be easily and rapidly secured within the medical device.

It is an advantage of particular embodiments of the present invention that the catheter is disposed about an incident angle of about 5-10°, in particular about 7° relative to the support.

An additional advantage of particular embodiments of the present invention is the possibility of removing and/or securing at least part of the medical article 24 (e.g. a medical tubing and/or a luer-lock of a catheter) without having to remove the medical article 24 in its entirety (e.g. catheter) from its fixed position within the medical device.

Therefore, to achieve these advantages, in a first aspect, the present invention relates to a medical device for securing a medical article to the skin of a patient, said medical article containing a luer-lock, the medical device comprising a deformable base element having a first surface allowing the base element to be tightly placed on a body area near the medical article application area, and a second surface opposite the first surface. The medical device further comprises a body member, comprising a first material and having a first side connected to the second surface of the base element, a second side opposite said first side, a front side, a back side opposite said front side and a pair of walls, characterized in that said medical device further comprises: a channel arranged at the second side of said body member and a clamping structure arranged at the second side of said body member; said channel being disposed about a longitudinal axis of the medical device, creating a passage extending from the front side to the back side of the body member and being shaped so as to receive at least a portion of the luer-lock. Said pair of walls at least partially enclosing said channel on both sides in order to support the luer-lock when the medical article is inserted in the medical device; wherein said clamping structure comprises a second material, the second material being less soft and deformable than the first material, said clamping structure cooperating with said channel to inhibit at least longitudinal and transversal movement of the medical article relative to said body member. Unless provided otherwise, the term “enclose” should be understood as to surround something in order to provide for support. For example, if a first object is enclosed by a second object, said first object is surrounded and supported by said second object.

In a preferred embodiment, the channel of the medical device according to the invention Comprises at least one groove oriented perpendicularly within said channel, wherein said clamping structure at least partially fits within said at least one groove, thereby cooperating with said channel to secure at least a portion of the luer-lock and inhibiting at least longitudinal and transversal movement of the medical article relative to the body member (this is for example illustrated in FIGS. 2-5).

In another embodiment, the pair of walls of the medical device according to the invention are inwardly tilted in order to generate a downward pushing force upon the medical article.

In a next embodiment, the channel of the medical device according to the invention further comprises at least one protrusion arranged at the inside of said channel, wherein the at least one protrusion cooperates with said pair of walls and said clamping structure to secure at least a portion of the luer-lock and inhibit at least longitudinal and transversal movement of the medical article relative to the body member.

In another preferred embodiment, the clamping structure of the medical device according to the invention is located at the front side of the channel or at the back side of the channel, the clamping structure cooperating with said channel to inhibit at least longitudinal and transversal movement of the medical article relative to the body member (this is for example illustrated in FIG. 6).

In a further preferred embodiment, the channel of the medical device according to the invention is angled relative to the longitudinal axis of the medical device to define an incident angle between the channel and the skin of the patient.

In another preferred embodiment, the incident angle of the medical device as defined herein, constitutes an angle of about 5-10°, in particular about 7°.

In a further preferred embodiment, the channel of the medical device as defined herein, has a concavity formed along at least a portion of the longitudinal axis of the medical device.

In yet another preferred embodiment, the channel of the medical device as defined herein, further includes a bottom wall, and the concavity extends upwardly from the bottom wall of the channel along a vertical axis of the channel.

In a next preferred embodiment, the channel of the medical device according to the invention has a longitudinal shape.

In a subsequent preferred embodiment, the channel of the medical device as defined herein further comprises a second groove oriented perpendicularly within said channel.

In a further preferred embodiment, the cooperation of the shape of the clamping structure and said channel of the medical device as defined herein allows different types of medical articles containing a luer-lock to be secured within said medical device.

In another preferred embodiment, the clamping structure of the medical device as defined herein comprises at least one clip assisting in clamping at least part of the medical article containing a luer-lock.

In yet another preferred embodiment, an outer rim of the luer-lock of the medical device according to the invention is secured within said second groove.

In a further preferred embodiment, the first surface of the deformable base element of the medical device as defined herein comprises an adhesive layer for removably coupling the medical device to the skin of a patient.

In another preferred embodiment, the pair of walls of the medical device as defined herein gradually decreases in height towards the outer side of said body member.

In a second aspect, the present invention relates to a kit comprising the medical device as defined herein and an adhesive sheet for at least partially covering the channel of the medical device when the medical article is secured therein.

Features from the dependent claims may be combined with features of the independent claims and with features of other dependent claims as appropriate and not merely as explicitly set out in the claims.

Although there has been constant improvement, change and evolution of devices in this field, the present concepts are believed to represent substantial new and novel improvements, including departures from prior practices, resulting in the provision of more efficient, stable and reliable devices of this nature.

The above and other characteristics, features and advantages of the present invention will become apparent from the following detailed description, taken in conjunction with the accompanying drawings, which illustrate, by way of example, the principles of the invention. This description is given for the sake of example only, without limiting the scope of the invention. The reference figures quoted below refer to the attached drawings.

### BRIEF DESCRIPTION OF THE DRAWINGS

FIG. 1 is an exploded view of a base element, a body member and a clamping structure, according to an embodiment of the present invention.

FIG. 2A is a top view of a medical device, according to an embodiment of the present invention.

FIG. 2B is a perspective view of a medical device, according to an embodiment of the present invention.

FIG. 3 is a schematic representation of a medical device securing at least part of a catheter, according to an embodiment of the present invention.

FIG. 4 is a side view of a medical device securing at least part of a catheter, according to an embodiment of the present invention.

FIG. 5 is a perspective view of a medical device, according to an embodiment of the present invention.

FIG. 6 is a perspective view of a medical device, according to an embodiment of the present invention.

FIG. 7A is a view of a top view, a bottom view, a left side view, a right side view, a front view and a rear view of a medical device, according to an embodiment of the present invention.

FIG. 7B is a vertical cross-sectional view of a medical device securing at least part of a catheter, according to an embodiment of the present invention.

FIG. 7C is a vertical cross-sectional view of a medical device securing at least part of a catheter, according to an embodiment of the present invention.

### DETAILED DESCRIPTION OF THE INVENTION

The present invention will be described with respect to particular embodiments and with reference to certain drawings, but the invention is not limited thereto. The drawings, as further described, are only schematic and are non-limiting. The dimensions and the relative dimensions do not correspond to actual reductions to practice of the invention.

Furthermore, the terms first, second, further and the like in the description and in the claims are used for distinguishing between similar elements and not necessarily for describing a sequence, either temporally, spatially, in ranking or in any other manner. It is to be understood that the terms so used are interchangeable under appropriate circumstances and that the embodiments of the invention described herein are capable of operation in other sequences than described or illustrated herein.

Moreover, the terms base, top, front, back and the like in the description and the claims are used for descriptive purposes and not necessarily for describing relative positions. It is to be understood that the terms so used are interchangeable under appropriate circumstances and that the embodiments of the invention described herein are capable of operation in other orientations than described or illustrated herein. It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a device comprising means A and B” should not be limited to devices consisting only of components A and B. It means that with respect to the present invention, the relevant components of the device are A and B, and that further components such as C may be present.

It is also to be noticed that the term “cooperating” used in the claims, for example component A cooperating with component B, should be interpreted as two components working together to ameliorate a certain effect. For example, in an embodiment of the current invention, the clamping structure cooperates (i.e. functions together) with the channel in order to inhibit movement of the medical article relative to the body member.

Besides that, when a part of the medical device is said to ‘inhibit’ movement of the medical article and unless defined otherwise, the term ‘inhibit’ should be interpreted as partially or completely reducing movement of the medical article. This term may alternatively be referred to as for example: “minimize”, “constrain” or “impede”.

Similarly, it is to be noticed that the term “removably coupled”, also used in the claims, should not be interpreted as being restricted to direct connections only. The terms “removably coupled” and “connected”, along with their derivatives, may be used. It should be understood that these terms are not intended as synonyms for each other. Thus, the scope of the expression “an element A removably coupled to an element B” should not be interpreted as elements (e.g. medical device, skin of a patient) being preferably inseparably attached to each other, but rather as a coupling which can be breached by means of a substantial pulling force. The term “connected” on the other hand, should be interpreted as describing an attachment between two elements (e.g. base element, body member) which is preferably inseparable. Thus, the scope of the expression “an element A connected to an element B” should be interpreted as an element A being preferably inseparably attached to element B.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment. Furthermore, the particular features, structures or characteristics may be combined in any suitable manner, as would be apparent to one of ordinary skill in the art from this disclosure, in one or more embodiments.

Similarly, it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some, but not other features included in other embodiments, combinations of features of different embodiments are meant to be within the scope of the invention, and form different embodiments, as would be understood by those in the art. For example, in the following claims, any of the claimed embodiments can be used in any combination.

In the description provided herein, numerous specific details are set forth. However, it is understood that embodiments of the invention may be practiced without these specific details. In other instances, well-known methods, structures and techniques have not been shown in detail in order not to obscure an understanding of this description.

The following terms are provided solely to aid in the understanding of the invention:

As used herein and unless otherwise specified, when a medical device is said to “secure a medical article to a support”, it is meant that at least a portion of the medical article is fixed to and/or stabilized to and/or positioned onto the support. Securing the medical article typically hinders, or even completely prevents, both translational and rotational movement of at least a portion of the medical article.

Besides that, as used herein and unless otherwise specified, the term “groove” is used to define a recess of the body member and more specifically of the channel, which is shaped so as to have the ability to accept different parts of the medical device (e.g. at least part of the clamping structure) and/or the medical article, (e.g. at least part of the medical article or luer-lock).

A first embodiment of the present invention relates to a medical device for securing at least part of a medical article to a support. The medical article may be a catheter, for example a central venous catheter, a peripheral venous catheter, a midline catheter, a continuous peripheral nerve block catheter or an epidural catheter. The medical device can advantageously be used in a plurality of applications.

In some embodiments, the support may be a body of a living creature. Securing the medical article to the living body advantageously reduces the risk of the medical article moving within the body or even being accidentally removed therefrom.

The medical device for securing a medical article to a support may comprise a base element having a first surface fixable to the support, e.g. living body, and a second surface opposite the first surface. It may also comprise a body member having a first side connectable with the second surface of the base element. According to embodiments of the present invention, while the first side of the body member is connected with the second surface of the base element in order to form a means for attaching the base element to a support, the second side of the body member defines a pair of walls and therebetween a channel for receiving the medical article. The body member further comprises a first material. The channel also comprises a clamping structure arranged at the second side of the body member. It was surprisingly found that the unique combination of the channel and the clamping structure may adapt to a relatively broad range of catheter sizes and catheter types, thus eliminating the need to buy and use several devices dedicated to specific catheter sizes and catheter types.

In some embodiments, the first side of the base element may be fixable to the support by means of an adhesive material. The adhesive material advantageously allows the first side to be easily fixed to the support. In preferred embodiments, the fixation may be reversible, such as by using a removable adhesive (e.g. tape, glue).

The channel is an integral part of the body member and therefore comprises the same first material as the body member. The first material generally being slightly flexible and deformable in order for the pair of walls to outwardly deform when receiving the medical article, but providing a sufficient amount of rigidness for the clamping structure in order to cooperate with the clamping structure in tightly securing the medical article within the channel.

In a preferred embodiment, the cooperation between the pair of walls and the clamping structure of the medical device as defined herein allows different types of luer-lock portions (of medical articles) to be sufficiently secured and inhibited within said medical device. More specifically, the pair of walls are sufficiently flexible so that, when a medical article is inserted, the pair of walls may outwardly deform, so that different types, sizes and diameters of luer-locks may fit within the channel of the medical device. Also, the pair of walls may provide for further inhibition of movement of the luer-lock because of the slight deformability of the pair of walls, increasing the contact surface between the luer-lock part of the medical article and the pair of walls.

Furthermore, the clamping structure cooperates with the pair of walls by being sufficiently rigid to enable a tight fixation and movement inhibition of the luer-lock within the medical article. This way, the securement of at least a portion of the luer-lock by the fixation mechanism of both the pair of walls and the clamping structure works synergistically in inhibiting movement of the medical article.

In some embodiments, the first material may be solid.

In some embodiments, at least part of the first material may comprise a deformable fluid filled chamber. The chamber may for example be filled with air or any other type of gas or with a fluid. In some embodiments, more than one channel may be defined within the body member of the medical device having similar features as the ones of the channel which is described in the present invention.

In the context of the present invention, the term “clamping structure” is meant to be a complementary part of the channel of the body member, which facilitates the unique securement of a number of different sizes and types of medical articles within the channel.

The clamping structure is generally made of a second material, being less flexible and deformable than the first material.

In some embodiments, the clamping structure may be unremovably coupled to the body member.

In some embodiments, the clamping structure and the channel are spaced apart from each other.

In some embodiments, the clamping structure may be separable from the body member, allowing a plurality of differently shaped clamping structures to be inserted into the channel of the body member.

In some embodiments, the clamping structure may be oriented at the front side of the channel or at the back side of the channel. In some embodiments, the clamping structure may comprise a number of additional materials other than the abovementioned second material. In preferred embodiments, the clamping structure is made solely of the second material.

In the context of the present invention, the term “clip” is meant to be an integral part of the clamping structure, having the ability to exert a clamping force upon at least part of the medical article.

In some embodiments, the clamping structure comprises a single clip which has the ability to exert a clamping force on at least part of the medical article.

In some embodiments, the clamping structure comprises at least one clip which has the ability to be outwardly deformed independently from the clamping structure.

In some embodiments, the clamping structure comprises a plurality of clips which all differ slightly in size and shape to be able to clamp a number of different parts of the medical article. In some embodiments, the at least one clip is generally U-shaped.

In some embodiments, the at least one clip is horseshoe shaped.

In some embodiments, the at least one clip is conically shaped.

In some embodiments, the at least one clip has a similar shape as the first groove and/or the further groove in which the clip may at least partially fit.

In some embodiments, the medical article may comprise a pair of wings which may be situated outside of the channel of the body member, more specifically at the front side of the body member.

A following embodiment of the present invention relates to the channel of the medical device, further comprising at least one groove oriented perpendicularly within said channel, wherein said clamping structure at least partially fits within said at least one groove thereby cooperating with said channel to secure at least a portion of the luer-lock and inhibiting or at least reducing longitudinal and transversal movement of the medical article relative to the body member.

In some embodiments, the channel may comprise at least one groove with a clamping structure contained therein, wherein the clamping structure may be in the form of an insert.

In some embodiments, the channel may define a single groove for receiving a clamping structure.

In some embodiments, the channel may comprise at least one groove which is not meant to receive the clamping structure but is in direct contact with at least part of the medical article.

In some embodiments, the channel may define two grooves. A first groove for receiving the clamping structure and a second groove for receiving at least part of the medical article (such as the luer-lock). In preferred embodiments, all of the grooves may be oriented perpendicularly relative to the longitudinal axis of the channel.

In some embodiments, the groove for receiving at least part of the medical article may be oriented parallel relative to the longitudinal axis of the channel.

In some embodiments, the grooves for receiving the clamping structure are oriented parallel relative to the longitudinal axis of the channel and the groove for receiving at least part of the medical article is oriented perpendicular relative to the longitudinal axis of the channel.

In some embodiments, all of the grooves may be oriented parallel relative to the longitudinal axis of the channel.

In some embodiments, the channel may define a single groove which is shaped in such a way to comprise all of the necessary properties for receiving the clamping structure and at least part of the medical article.

In some embodiments, all of the at least one groove may be for example only present in the bottom wall of the channel while only part of the clamping structure is contained within the at least one groove.

In some embodiments, the clamping structure may be contained within the channel while the channel does not define any grooves.

In some embodiments, the clamping structure mainly inhibits the longitudinal movement of the medical article relative to the body member.

In some embodiments, the clamping structure inhibits both longitudinal and transversal movement of the medical article relative to the body member.

In some embodiments, the clamping structure mainly inhibits the transversal movement of the medical article relative to the body member.

In another embodiment, the pair of walls of the medical device according to the invention are inwardly tilted in order to generate a downward pushing force upon the medical article.

The fact that the pair of walls are inwardly tilted generates a partial enclose of at least a portion of the luer-lock, causing a downward pushing force upon said portion. Because of this downward pushing force, said luer-lock portion is tightly secured within the channel of the medical device.

An advantage of embodiments of the current invention wherein the pair of walls are inwardly tilted is that the luer-lock portion which is held by the pair of walls is unable to move upwardly, being in the direction of the second side of the body member.

As mentioned herein and unless provided otherwise, the “inwardly tilted” pair of walls should be understood as a being a pair of walls that point towards each other at least with the end of each wall. This way, the pair of walls are not oriented parallel relative to each other but rather create a converging shape (e.g. horseshoe shape or conical shape) towards the end of the pair of walls. Because of this inwardly tilting motion, the inwardly tilted ends of each of said pair of walls exert a downward pushing force at least upon the top side of said luer-lock portion.

In a next embodiment, the channel of the medical device according to the invention further comprises at least one protrusion arranged at the inside of said channel, wherein the at least one protrusion cooperates with said pair of walls and said clamping structure to secure at least a portion of the luer-lock and inhibit at least longitudinal and transversal movement of the medical article relative to the body member.

In some embodiments, said protrusion may be deformable. increasing the contact surface between the luer-lock part of the medical article and the protrusion.

In some embodiments, at least part of the protrusion may comprise a deformable fluid filled chamber. The chamber may for example be filled with air or any other type of gas or with a fluid.

A following embodiment of the present invention relates to the clamping structure of the medical device, being located at the front side of the channel or at the back side of the channel. The clamping structure cooperating with said channel to inhibit at least longitudinal and transversal movement of the medical article relative to the body member.

In some embodiments, the clamping structure is located directly in front of the channel.

In some embodiments, the clamping structure is located directly behind the channel.

In some embodiments, the clamping structure and the channel are lined up according to a longitudinal axis of the medical device.

In some embodiments, the diameter of the clamping structure differs from the diameter of the channel.

Another embodiment of the present invention relates to the channel of the medical device, being angled relative to the longitudinal axis of the medical device to define an incident angle between the channel and the skin of the patient. The incident angle constituting an angle of about 5-10°, in particular about 7°. The angle of about 7° provides for an anti kinking feature for the catheter, thereby providing for an enhancement of the success rate of a correct catheter insertion of the medical article into the skin of the patient.

In the context of the present invention, the “angle” is formed between at least part of the catheter of the medical article and the skin of the patient.

In some embodiments, the angle may be slightly less than 5°. In other embodiments, the angle may be slightly more than 10°. In preferred embodiments, the angle may be approximately 7°.

A fifth embodiment of the present invention relates to the channel of the medical device having a concavity formed along at least a portion of the longitudinal axis of the medical device. The channel further including a bottom wall, the concavity extending upwardly from the bottom wall of the channel along a vertical axis of the channel and the channel having a longitudinal shape.

In some embodiments, the concavity is formed along the entire length of the longitudinal axis of the medical device.

In some embodiments, the channel of the medical device does not define a concavity, but a multangular shape.

In some embodiments, at least part of the channel has a circular shape.

In some embodiments, at least part of the channel has an angular shape.

A next embodiment of the present invention relates to the channel of the medical device comprising a second groove oriented perpendicularly within said channel.

In some embodiments, the second groove is oriented parallelly within the channel.

In a following embodiment of the present invention, the cooperation between the shape of the clamping structure, the channel and the second groove is described, allowing different types of medical articles, in particular containing a luer-lock, to be secured within the medical device. In the context of the present invention, the term “cooperation” indicates the utilization of the combined functionalities of multiple different shapes and features of different parts of the medical device in order to tightly secure at least part of the medical article within the medical device.

In some embodiments, the cooperation exists only between the shape of the clamping structure and the channel.

In some embodiments, part of the luer-lock of the medical article is located outside the channel of the body member.

A further embodiment of the present invention relates to the clamping structure comprising at least one clip assisting in clamping at least part of the medical article containing a luer-lock.

In the context of the present invention, the term “clip” refers to a part of the clamping structure having the ability of at least partially enclosing at least part of the medical article.

In some embodiments, the clamping structure comprises a single clip.

In some embodiments, the at least one clip is at least partially fitted within the at least one groove.

In some embodiments, the at least one clip has a similar shape as the groove in which it is at least partially fitted.

In some embodiments, the clip comprises at least two opposing posts. The at least two opposing posts may contact at least part of the luer-lock of the medical article.

In some embodiments, the clip comprises a single post.

A next embodiment of the present invention relates to the second groove of the medical device having an outer rim of the luer-lock secured therein.

In the context of the present invention, the term “rim” refers to an edged part of the luer-lock of the medical article.

In some embodiments, only part of the outer rim may be fitted within the second groove.

A further embodiment of the present invention relates to the first surface of the deformable base element of the medical device, comprising an adhesive layer for removably coupling the medical device to the skin of a patient.

In some embodiments, the adhesive layer may cover only part of the first surface of the deformable base element.

A next embodiment of the present invention relates to the pair of walls of the medical device, which gradually decrease in height towards the outer side of the body member.

In some embodiments, the pair of walls do not change in height throughout the entire width of the body member.

In some embodiments, the pair of walls do not change in height throughout the entire length of the body member.

Another embodiment of the present invention relates to a kit comprising the medical device and an adhesive sheet for at least partially covering the channel of the medical device when the medical article is secured therein.

In some embodiments, the adhesive sheet covers the entire surface of the medical device.

In some embodiments, the adhesive sheet covers the entire body member of the medical device.

In some embodiments, the adhesive sheet covers at least part of the channel and the medical article, which is secured therein.

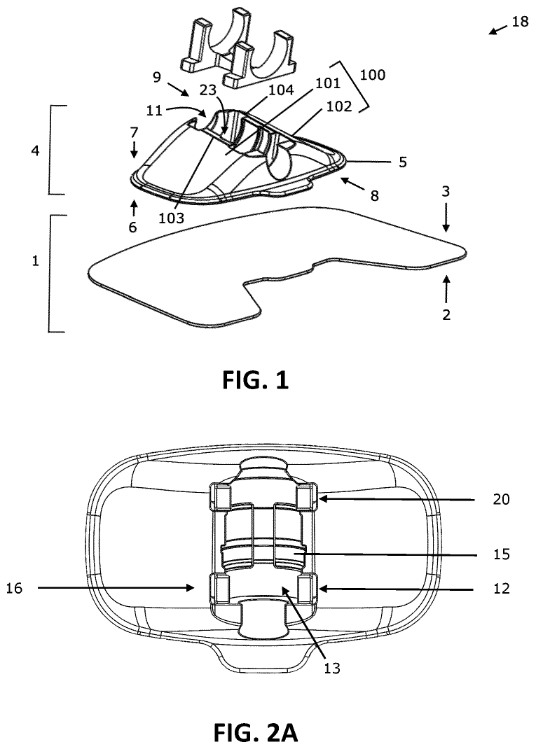

We now refer to FIG. 1, showing an exploded view of a medical device 18 in accordance with the present invention. The medical device 18 comprises a base element 1 and a body member 4. In this example, the base element 1 comprises a first surface 2 for securing the medical device 18 to a support (e.g. an arm or a leg of a patient) and a second surface 3 opposite the first surface 2. The base element 1 may for example be flexible such that it can conform to the shape of the support and may be removably coupled to the support using an adhesive layer 17 (e.g. a tape) which is connected to the first surface 2 of the base element 1. The body member 4 may comprise a first side 6 which is connected to the second surface 3 of the base element 1, a second side 7 opposite the first side 6 for receiving at least a portion of a number of different types and different sizes of a medical article containing a luer-lock 24 (hereinafter: ‘medical article’ 24), a front side 8, a back side 9 opposite said front side 8 and a pair of walls 100. The body member 4 comprises a first material 5 which allows the body member 4 to at least partially conform to the support. The first material 5 provides a sufficient amount of flexibility to the body member 4, such that the pair of walls 100 are able to slightly deform outwardly when securing the medical article. Besides that, the first material 5 allows the body member 4 to slightly deform in its entirety, for example when the patient is applying pressure on the medical device 18, which may be achieved by lying on the medical device 18. Because of this feature, the patient will experience noticeably less discomfort compared to medical devices consisting of stiff, non-deformable materials. Moreover, the pair of walls 100 may gradually decrease in height towards the outer sides of the body member 4, creating a sloping second side 7 of the body member 4. The body member 4 is typically fixed to the base element 1 and defines a channel 11 located at the second side 7 of the body member 4, being of sufficient size so as to receive the medical article 24 at the second side 7 of the body member 4. The channel 11 has a concave shape and a longitudinal shape, which may occupy the entire area between the first wall 101 and the second wall 102 while the pair of walls 100 are at least partially enclosing the channel 11 in order to further support the medical article 24. The top side 103 of the first wall 101 and the top side 104 of the second wall 102 may be the outer ends of the channel 11. The channel 11 is disposed about a longitudinal axis of the medical device 18, creating a passage which extends from the front side 8 to the back side 9 of the body member 4. The channel 11 further includes a bottom wall 23. The concave shape extending upwardly from the bottom wall 23 of the channel 11 along a vertical axis of the channel 11.

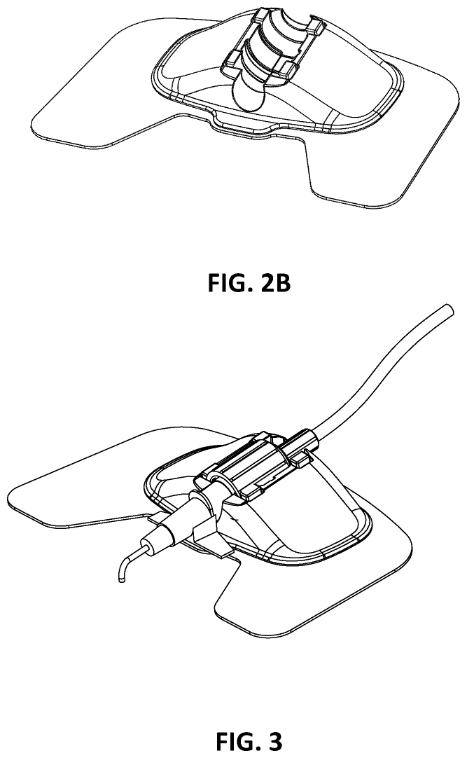

We now refer to FIG. 2A, showing a top view of an assembled version of the different elements of the medical device 18 as described in the detailed description relating to FIG. 1. The channel 11 may further comprise the at least one groove 12 (the at least one groove 12 may further be referred to as the first groove 12) being oriented perpendicularly relative to the longitudinal axis of said channel 11. The first groove 12 may reach from the first wall 101 to the second wall 102 and may be horeshoe shaped. The channel 11 may also comprise a further groove 20 having generally the same properties as the first groove 12 and being oriented perpendicularly relative to the longitudinal axis of the channel 11. The first groove 12 and the further groove 20 being spaced apart within the channel 11.

The body member 4 further comprises a clamping structure 13 (such as in the form of an inset) which is shaped so as to at least partially fit within the first groove 12 and the further groove 20.

The clamping structure 13 may comprise at least one clip 16 made of a second material 14. The second material 14 being less soft and deformable than the first material 5 and being sufficiently rigid so that the at least one clip 16 has the ability to cooperate with the channel 11 in securing the medical article 24. The at least one clip 16 preferably clamping the medical article 24 in order to assist in inhibiting at least longitudinal movement of the medical article 24 relative to the body member 4. The at least one clip 16 may further have a similar horseshoe shape as the first groove 12. The channel 11 may further comprise a second groove 15 oriented perpendicularly relative to the longitudinal axis of the channel 11. The second groove 15 cooperates with the clamping structure 13 and the channel 11 in order to allow different shapes and types of medical articles containing a luer-lock 24 to be secured within said medical device 18. More specifically, an outer rim of the luer-lock may be secured within said second groove 15. Furthermore, the second groove 15 may have a similar horseshoe shape as the first groove 12, assisting in the securement of at least part of the medical article 24. The second groove 15 thereby inhibiting at least longitudinal movement of the medical article 24 relative to the body member 4. The interplay between the clamping structure 13, and the channel of the body member 4 is an unique feature of the present invention, allowing a number of different shapes and sizes of medical articles 24 to be tightly secured into place. This unique feature further allows for the rapid and simple securement of the medical article 24 within the medical device 18, which is an important advantage of the present invention.

In this particular embodiment, the clamping structure 13 is at least partially fitted within the first groove 12 and the further groove 20, thereby generally conforming to the overall shape of the channel 11 and allowing further securement of the medical article.

We now refer to FIG. 2B, showing a perspective view of an assembled version of the different elements of the medical device 18 as described in the detailed description relating to FIG. 1. The channel 11 is angled relative to the longitudinal axis of the medical device 18. More specifically, an incident angle of about 5-10°, in particular about 7°, between the channel 11 and the skin of the patient can be defined, creating an optimal angle for the medical article 24 when puncturing the skin of the patient with at least part of the medical article 24. The channel 11 defines a first opening 22A positioned at the front side 8 of the body member 4 and a second opening 22B positioned at the back side 9 of the body member 4. Both openings 22A, 22B act as the outer ends of the channel 11 and assist in the fixation of the medical article 24. An adhesive sheet may be applied on top of the body member 4 for at least partially covering the channel 11 of the medical device 18 when the medical article 24 is secured. This adhesive may further assist in the securement of the medical article 24 within the medical device 18.

We now refer to FIG. 3, showing a medical device 18 securing a medical article 24, more specifically a medical article comprising a luer-lock and a pair of wings 25. The first side 6 of the body member 4 is attached to the second surface 3 of the base element 1. In this embodiment, the clamping structure comprises 2 separate clips 16A, 16B. The clips 16 of the clamping structure 13 both exert a clamping force upon at least part of the medical article 24 and the second groove 15 of the body member 4 assists in the fixation of the medical article 24. An easy securement of the medical article 24 is provided for, without having to exert a great amount of pressure. To this end, the clips 16 and the pair of walls 100 of the body member 4 are being slightly outwardly deformed when introducing the medical article 24. The pair of walls 100 provide an additional support at both sides of the medical article 24. The pair of wings 25 are located directly at the front side 8 of the body member 4 and, consequently, at a peripheral position relative to the channel 11 of the body member 4. The medical article 24 is disposed at an angle of about 7° relative to the support. The base element 1 comprises a recess in the middle 26 and at the front side 8 of the body member 4 so that it does not come in contact with the medical article 24. The medical article 24 is introduced in the channel 11 at the second side of the body member 4, while the bottom wall 23, the pair of walls 100, the first opening 22A, the second opening 22B and the clamping structure 13 support the at least part of the medical article 24 thereby inhibiting at least longitudinal movement of the medical article 24 relative to the body member 4.

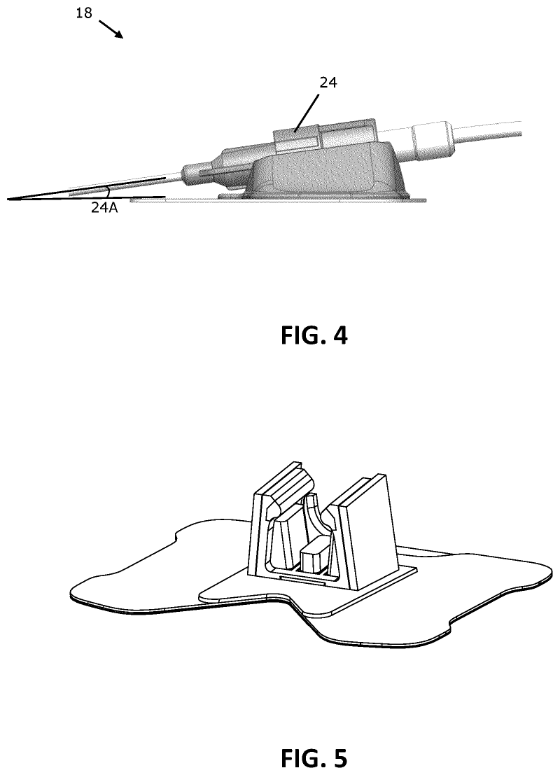

We now refer to FIG. 4, showing a side view of a medical device 18 securing a medical article 24, more specifically at least part of the medical article 24 being disposed at an angle of about 7° relative to the support. The angle of the catheter 24A minimizes the chance of complications due to the introduction of the catheter in the support (e.g. the skin of a patient).

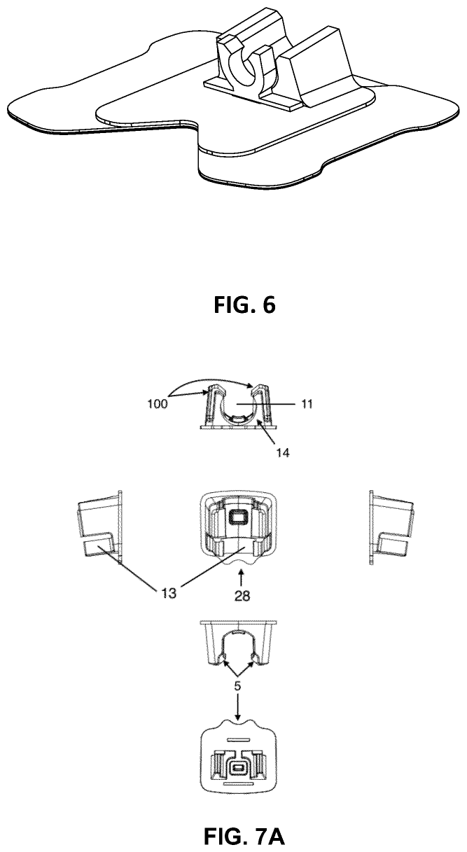

We now refer to the medical devices disclosed in FIG. 5 and FIG. 6, which both have a similar basis structure compared to the one as disclosed in anyone of FIGS. 1 to 4. Therefore, a number of similar structural elements will be indicated using the same numbers as in anyone of FIGS. 1 to 4 and structural elements deviating with respect to function and/or shape will be described in more detail.

We now refer to FIG. 5, showing a perspective view of a medical device 18, according to an embodiment of the present invention.

The clamping structure 13 comprises a number of protrusions 27, which are spaced apart from each other and defined at the inside of the clamping structure 13. The protrusions 27 may comprise a deformable solid material, being able to adapt to different catheter sizes and types, such as the material of the body member 4. More specifically, the protrusions 27 may aid in the fixation and movement inhibition of different types and sizes of luer-locks.

Besides that, the protrusions 27 cooperate with the clamping structure 13 and the body member 4 in order to inhibit at least longitudinal and transversal movement of the medical article 24 relative to the body member 4. The contact between the protrusions 27 and at least part of the medical article 24 (e.g. part of the luer-lock) is of such a nature that a certain resistance arises which inhibits at least the longitudinal movement of the medical article 24. More specifically, a certain resistance may be accomplished when the luer-lock is inserted in the medical device and at least one of the protrusions 27 is deformed by at least a portion of the luer-lock, which increases the contact surface between both the luer-lock and the at least one protrusion 27. This advantage of increased contact surface is especially useful when fixating luer-locks with a ribbed outer surface. Moreover, further resistance may be accomplished when at least one of the protrusions 27 is able to hook behind, in front or above the luer-lock.

Some parts of the body member 40 are shaped in such a way to further define the conically shaped channel 11 and to enhance the securement and inhibition of movement of at least part of the medical article 24 relative to the body member 4 in a similar way as the protrusion 27 do in this particular embodiment.

The clamping structure 13, surrounding the protrusions 27 and some parts of the body member 40, complements the effect of the protrusions 27 and the body member 4 by further inhibiting the longitudinal and transversal movement of the medical article 24. The clamping structure may contact different parts of the luer-lock then those parts contacting the protrusion 27 in order to facilitate this complementary effect.

We now refer to FIG. 6, showing a perspective view of a medical device, according to an embodiment of the present invention. The clamping structure 13 is located at the front side 28 (or alternatively the back side) of the channel 11. The diameter of the clamping structure 13 may be smaller than the diameter of the channel 11. The clamping structure 13 exerting a clamping force upon a part of the medical article 24, inhibiting at least the longitudinal movement of the medical article 24. The channel 11 inhibiting at least the transversal movement of the medical article 24. Both the clamping structure 13 and the channel 11 cooperating in order to secure the medical article 24 to the support.

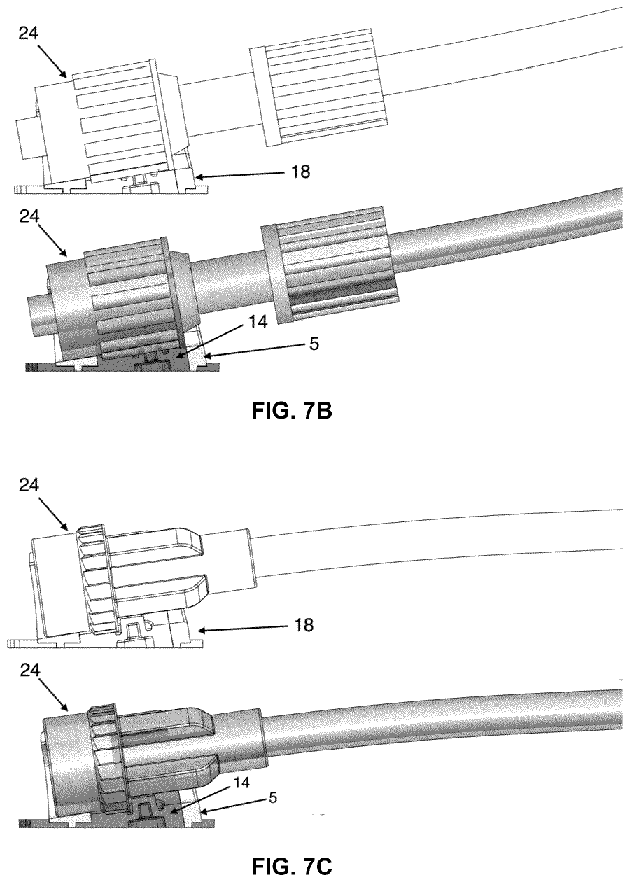

We now refer to FIG. 7A, showing a top view, a bottom view, a left side view, a right side view, a front view and a rear view of a medical device, according to an embodiment of the present invention. The clamping structure 13 is located at the front side 28 (or alternatively the back side) of the channel 11 and lies in line therewith. The first material 5 of the body member 4 cooperates with said pair of walls 100 in order to tightly secure the medical article within the medical device. FIG. 7B is a vertical cross sectional view of a medical device securing at least part of a catheter, according to an embodiment of the present invention. In this figure, it is clearly shown how the luer-lock portion of a medical article 24 is secured within the medical device 18.

FIG. 7C is a vertical cross sectional view of a medical device securing at least part of a catheter, according to an embodiment of the present invention. The same elements are shown as in FIG. 7B, although another type of luer-locks is secured within the medical article. In this case, it is clearly shown that only part of the luer-lock portion of the medical article 24 is secured by the medical device 18. In both FIGS. 7B and 7C, the cooperation between the first material 5 and the second material 14 in order to secure the medical article 24 is clearly visible.

1-17. (canceled)

18. A medical device for securing a medical article having a luer lock to the skin of a patient, the medical device comprising:
a deformable base element having a first surface allowing the base element to be tightly placed on a body area near an application area of the medical article, and a second surface opposite the first surface;
a body member comprising a first material and having a first side connected to the second surface of the base element, a second side opposite the first side, a front side, a back side opposite the front side, and a pair of walls;
a clamping structure arranged at the second side of the body member; and
a channel arranged at the second side of the body member the channel being disposed about a longitudinal axis of the medical device, creating a passage extending from the front side to the back side of the body member and being shaped so as to receive at least a portion of the luer-lock,
wherein:
the pair of walls of the body member at least partially encloses the channel on both sides of the channel to support the luer-lock when the medical article is inserted in the medical device; and
the clamping structure comprises a second material, the second material being less soft and deformable than the first material, the clamping structure cooperating with the pair of walls to inhibit at least longitudinal and transverse movement of the medical article relative to the body member.

19. The medical device of claim 18, wherein:
the channel further comprises at least one groove oriented perpendicularly within the channel; and
the clamping structure at least partially fits within the at least one groove thereby cooperating with the channel to secure at least a portion of the luer-lock and inhibiting at least longitudinal and transversal movement of the medical article relative to the body member.

20. The medical device of claim 18, wherein the pair of walls are inwardly tilted to generate a downward pushing force upon the medical article.

21. The medical device of claim 18, wherein:
the channel further comprises at least one protrusion arranged at the inside of the channel; and
the at least one protrusion cooperates with the pair of walls and the clamping structure to secure at least a portion of the luer-lock and inhibit at least longitudinal and transversal movement of the medical article relative to the body member.

22. The medical device of claim 18, wherein:
the clamping structure is located at the front side of the channel or at the back side of the channel; and
the clamping structure cooperates with the channel to inhibit at least longitudinal and transversal movement of the medical article relative to the body member.

23. The medical device of claim 18, wherein the channel is angled relative to the longitudinal axis of the medical device to define an incident angle between the channel and the skin of the patient.

24. The medical device of claim 23, wherein the incident angle constitutes an angle of about 5° to 10°.

25. The medical device of claim 18, wherein the channel has a concavity formed along at least a portion of the longitudinal axis of the medical device.

26. The medical device of claim 25, wherein:
the channel further includes a bottom wall; and
the concavity extends upwardly from the bottom wall of the channel along a vertical axis of the channel.

27. The medical device of claim 26, wherein the cooperation of the shape of the clamping structure and the channel allows different types of medical articles containing a luer-lock to be secured within the medical device.

28. The medical device of claim 18, wherein the channel has a longitudinal shape.

29. The medical device of claim 18, wherein the channel further comprises a second groove oriented perpendicularly within the channel.

30. The medical device of claim 18, wherein the clamping structure comprises at least one clip assisting in clamping at least part of the medical article containing a luer-lock.

31. The medical device of claim 18, wherein an outer rim of the luer-lock is secured within the second groove.

32. The medical device of claim 18, wherein the first surface of the deformable base element comprises an adhesive layer for removably coupling the medical device to the skin of a patient.

33. The medical device of claim 18, wherein the pair of walls of the body member gradually decreases in height toward the outer side of the body member.

34. A kit comprising:
the medical device according to claim 18; and
an adhesive sheet for at least partially covering the channel of the medical device when the medical article is secured therein.
